(19) United States
(12) Patent Application Publication
(10) Pub. No.: US 2010/0215705 A1
(43) Pub. Date: Aug. 26, 2010

(54) Use of Colloidal Clays for Sustained Release of Active Ingredients

(75) Inventors: Dominic A. Cataldo, Kennewick, WA (US); Edward S. Lipinsky, Fairview, NC (US); Peter VanVoris, Daniel Island, SC (US)

Correspondence Address: MUELLER AND SMITH, LPA;MUELLER-SMITH BUILDING, 7700 RIVERS EDGE DRIVE, COLUMBUS, OH, 43235, US

(73) Assignee: BIOGUARD RESEARCH AND DEVELOPMENT, INC., Kennewick, WA (US)

(21) Appl. No.: 12/749,078

(22) Filed: Mar. 29, 2010

Related U.S. Application Data
(63) Continuation-in-part of application No. 10/816,095, filed on Apr. 1, 2004, now abandoned.
Application No. 10/816,095 is a continuation-in-part of application No. 10/325,327, filed on Dec. 20, 2002, now abandoned.
(60) Provisional application No. 60/543,184, filed on Feb. 10, 2004.

(51) Int. Cl. A01N 25/30; A61K 47/02; A61K 47/30; A01P 3/00; A01P 17/00; A01P 19/00

(52) U.S. Cl. 424/405; 514/770; 514/772

(57) ABSTRACT

One aspect of the invention is a method of controlling target living species with an active ingredient over an extended period of time. This method sorbs an active ingredient into colloidal clay. Next, a polymer pellet recalcitrant to release of the active ingredient is loaded with the sorbed colloidal clay. Finally, the loaded polymer pellet is formed into a barrier, which is adapted to be placed at a location for controlling a target living species. Another aspect is a barrier for controlling a target living species. The formed barrier contains polymer pellets loaded with colloidal clay sorbed with an active ingredient that controls the target living species. The sorbed clay loaded polymer pellets are recalcitrant to release of the active ingredient.

### CROSS-REFERENCE TO RELATED APPLICATIONS

This application is a continuation-in-part of application Ser. No. 10/816,095, filed on Apr. 1, 2004; which is a continuation-in-part of application Ser. No. 10/325,327, filed Dec. 20, 2002, and is cross-referenced to application Ser. Nos. 10/438,559, filed May 15, 2003; 10/698,722, filed Oct. 31, 2003; and 60/543,184, filed Feb. 10, 2004; the disclosures of which are expressly incorporated herein by reference.

### STATEMENT REGARDING FEDERALLY SPONSORED RESEARCH

Not applicable.

### BACKGROUND OF THE INVENTION

The present invention generally relates to formulations that release pesticides, repellents and attractants for insects and other animals, fungicides, herbicides, repellents for plant roots, and like active ingredients (control agents) and more particularly to the use of colloidal clays for sustained and controlled release of such active ingredients.

### Herbicides and the Like

Roots from trees and shrubs are known to cause a variety of problems as well as damage to man-made infrastructure. For example, sanitary and storm drain systems in cities and other municipalities are aging with part of the problem induced by the roots of trees. The aging process involves physical cracks and joint dislocations, resulting in leakage of sewage and storm waters to soils, ground waters, and surface waters. A number of remedial relining methods are available. These work for a short period and are then degraded by plant roots seeking out moisture and nutrients, entering the lines, dislocating and degrading the linings, and thus creating the original problem. To counter this problem of plant-root intrusion, options have been used whereby liquid herbicide is simply flushed through the sewer lines or the sewer line is wrapped with a herbicide releasing fabric; however, the later needs to be done when the sewer line has been dug up for repairs or when new sewer lines are being laid.

New products and methods are needed to control the intrusion of roots not only into sewer and other pipes, but also into sidewalks, golf course areas, especially greens. The new methods and products also could be used to control weed growth in landscaping, plant nursery, and agricultural situations.

For piping systems, these methods and products should offer reduced transport of herbicide into the environment and reduced disturbance of the piping system. For sidewalks, golf courses, landscapes, etc., application of the product should disrupt the system rarely.

Noxious weeds, including, for example, several species of knapweed and leafy spurge, continue to plague farmers, ranchers, and wildlife in the western United States. An improved weed control system is needed that will provide long term control while reducing pollution and environmental damage. Recent advances in pesticide formulations may offer a solution to this problem. A sustained release delivery system capable of delivering small but continual doses of herbicide to the weeds may provide the long term control that farmers and natural resource managers need. Sustained release formulations can deliver the most effective chemical concentration while not saturating the environment or expose humans and wildlife to high concentrations of chemicals. These specialized formulations can deliver a smaller total quantity of chemicals than the traditional broadcast and spraying methods used to apply commercial herbicides resulting in safer and more effective control of noxious weeds for farmers, ranchers, and natural resources managers.

### Insecticides, Repellents, and Attractants

Wood and wood products utilized in a variety of construction applications are frequently structurally degraded by the action of termites, ants, other boring insects, and wood decaying microorganisms. Typically, these wood degrading and decaying organisms migrate to wood structures via the surrounding soil or water. This migration may occur whether the structures rest upon concrete foundations, such as in wooden building construction; are in direct contact with the soil, for example fence posts, utility poles, railroad cross-ties, wooden supports, and like structures; or are in the water, such as boats, piers, pier pilings, wooden docks, or other supports. Wood and wood-containing products include, inter alia, glued wood products such as, for example, plywood, particleboard, oriented strand board (OSB), medium density fiberboard (MDF), laminated veneer lumber (LVL), laminated beams, and a variety of other engineered wood products. Paper products (especially paperboard and kraft paper) also are subject to degradation by organisms that attack wood. Outdoor furniture also is subject to wood degrading and decaying organisms. In the marine context (including for example, pleasure and commercial craft for use on lakes, rivers, and oceans), the structures additionally may be manufactured from fiberglass, various plastics, metals, ceramics, and other materials.

Present methods of preventing or retarding the advance of these wood degrading organisms include soil treatment with pesticides and repellent chemicals, treatment of the wood with chemicals, and fumigation wherein the entire structure may be sealed and a pesticide pest repellent released. Both soil and fumigation type treatments may release the pesticide to the surrounding atmosphere and/or the pesticide may move to ground water where it may harm human beings or other living organisms. Disadvantages of these methods of treating soil and/or fumigating include, inter alia, potential ecological and human health concerns, as well as the limited time until the fumigant or soil concentration is sufficiently reduced in concentration to permit ingress of wood degrading organisms.

Although many pesticides and repellents are known to be effective against the action of wood destroying organisms, their effectiveness often declines over time as they are dissipated into the surrounding environment (soil, water, or atmosphere) or are degraded, for example, chemically or biologically. To retain their effectiveness, these insecticides must be repeatedly applied at regular intervals ranging from every few days to every few months to every few years. Alternatively, if the pesticides and repellents are applied in sufficient quantity to be effective over an extended period of time, the ecological and human health related concerns associated with these chemicals and their unpleasant odors are exacerbated. Furthermore, with the banning of certain chemicals and the introduction of safer shorter half-life compounds, even large amounts of many of these pesticides and repellents may be required over a relatively short time periods, and they will need to be reapplied more often.

A further disadvantage of conventional application methods is that the concentration of pesticides and repellents resulting from a single application starts out well above the minimum concentration necessary for effectiveness, but decreases rapidly. Within a relatively short period of time the concentration drops below the minimal effective level necessary to maintain a barrier to the invasion of wood compromising organisms.

An important strategy in control of insects and unwanted animals is the use of attractants. Volatile bait can be employed to bring the target animal to a device that kills it. This method is especially useful for flying insects. The bait method also can be used to measure the identity of species that are in a vulnerable area and to quantify the intensity of an insect infestation

### General

Though prevention of unwanted plant growth and unwanted animals would seem to be unrelated, both areas have certain common goals. One such goal is to be able to release the active ingredient at a desired location at a desired target and at a lower, yet effective concentration. Another goal is to release the active ingredient at a desired or target concentration. A further goal is to release the active ingredient over an extended period of time. The invention addresses each of these goals by providing a mechanism whereby sufficient active ingredient is stored within pellets for release of a target concentration of active ingredient over an extended periods of time ranging from days to weeks to years and even up to several decades.

### BRIEF SUMMARY OF THE INVENTION

One aspect of the invention is a method of controlling target living species with an active ingredient (or control agent) over an extended period of time. In this method, an active ingredient is sorbed into colloidal clay. The resulting product may be used as-is to control the target living species. Next, a polymer pellet is loaded with the active ingredient loaded colloidal clay. The resulting product may be used as-is to control the target living species. Finally, the loaded polymer pellet is formed into a device that is adapted to be placed at a location for controlling a target living species. The formed device contains polymer pellets loaded with colloidal clay sorbed with an active ingredient that controls the target living species. The sorbed colloidal clay-loaded polymer pellets are recalcitrant to release of the active ingredient.

The active control ingredient will be “liquefied” for its sorbing by the nanoclay. By “liquefied” or “fluid” or “fluent” is meant that the (normally) solid active control ingredient will be heated to be fluent, or a vapor, so that in the liquefied state, the colloidal clay will adsorb the control agent. Such heating will be to a temperature of at least about 10° C. above the melting point of the solid active control agent on up to about 30° C. above the melting point of the solid active control agent. By “solid active control agent”, we mean that the active control agent is a solid at room temperature. At lower temperatures, the active control agent tends to not be absorbed by the nanoclay and/or deposits on the outer surface of the nanoclay, thus clogging the porosity of the nanoclay and preventing further absorption of the active control agent by the nanoclay or colloidal clay. The addition of an active control agent dissolved in a solvent (even at saturation), has a similar effect. The clay does load easier, but on evaporation of the solvent, much of the active control agent ends up diffusing out of the intercalated clays, and crystallizing on the outside of the clay. Additionally, the loading rate is inherently lower than disclosed method. Finally, loading of the nanoclay should not be excessive as a non-friable (gummy) mixture can result.

For these same reasons, the nanoclay also should be heated prior to its being contacted by the heated active control agent. It is best if the nanoclay is heated to at least same temperature, as is the active control agent to maximize absorption of the heated active control agent by the nanoclay, and the nanoclay can be heated to a temperature of at least about 15° C. above the melting point of the solid active control agent.

In practice, it is advantageous to heat the nanoclay or other absorbing carrier as high as feasible without thermally decomposing and/or loosing too much due to the active control agent vapor pressure. With small 10-20 gm samples, one can do the mixing quickly and uniformly at the lower temps. With 0.1 kg and above batches, the clay is normally heated to >15° C. above the melting point of the active control agent, and the active control agent can be amended at some temperature above its melting point (>1°-2° C.), but in practice >5°-10° C. can be used, which allows slow mixing and adsorption, without too much surface crystallization and agglomeration of clay/absorbent particles. This all becomes evident when you can load to >30% (w/w) without the clay carrier clumping (remains friable).

While the terms “colloidal clay” and “nanoclay” will be used herein, perhaps, a more technically accurate description of such materials would be “intercalated phyllosilicate” or “intercalated layered silicate”. For present purposes, these terms are used interchangeably and are equivalent in meaning. Clay is an aggregation of individual platelets. “Exfoliation” is the term for separation of the aggregation into individual platelets. “Tactoid” is the term for small stacks of clay platelets. In this invention, intercalation, tactoids, and exfoliation occur when the colloidal clay is chemically modified by prior treatment with onium amine compounds.

“Recalcitrant” to release of the active ingredient means that the loaded polymer pellets retard the release of the active ingredient to provide a sustained release over time. Appropriate times can be years to decades for some target species and days to months for other target species. The inventive sorbed colloidal clay-loaded polymer pellets can be designed or tailored to meet the demand requirements of a variety of pests.

Polymer “pellets” means particulates, discrete or agglomerated, regardless of shape—smooth, rough, jagged, or the like. Pellets, then, can be described as, for example, beads, particles, grains, crumbs, bits, or the like. Again, shape is unimportant with size determined by intended use in terms of environment, target species, type of control agent, type of barrier, and like factors.

“Target living species” or “target species” means any living organism including, inter alia, plants, animals, fungi, bacteria, viruses, insects, fish, mollusks, and the like. Target species can be found anywhere, including, inter alia, in the air, under the ground, on the ground, in water, in/on structures (both living and inanimate), or anywhere else.

“Control agent” or “active ingredient” means a chemical and/or biological agent that has the function of controlling a target species. In turn, “control” means to repel, attract, kill, or exert a desired action on a target species.

### BRIEF DESCRIPTION OF THE DRAWING

FIG. 1 diagrammatically depicts a method for loading nanoclay with the active ingredient and then forming a variety of products for controlling target living species. The drawing will be described in detail below.

### DETAILED DESCRIPTION OF THE INVENTION

The control products or devices of this invention can take the form of a barrier to prevent infestation of a specific location or an attractor that lures the target species to a specific location or a signal that affects the behavior of the target living species.

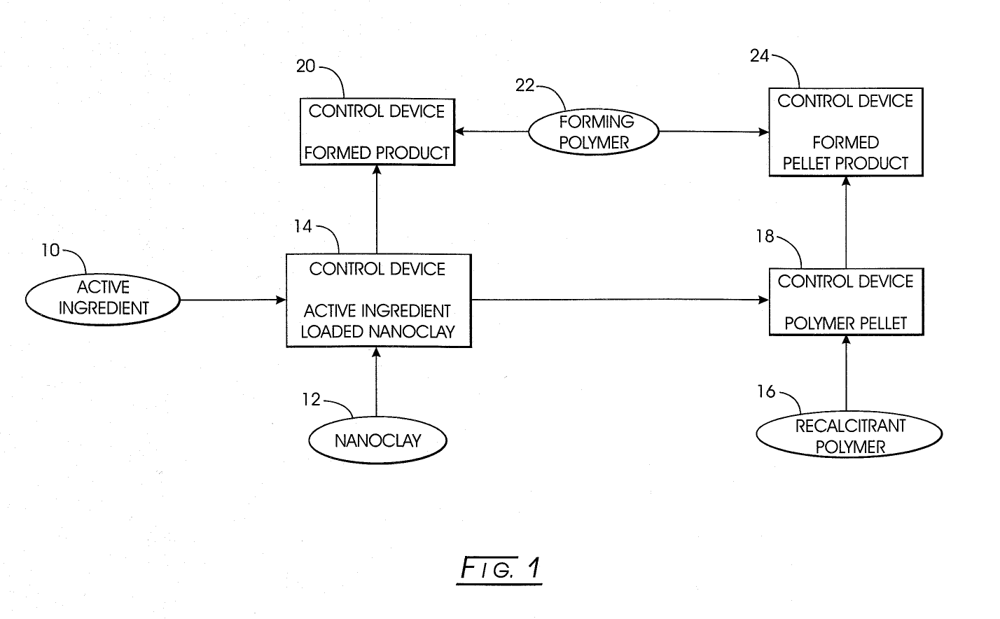

FIG. 1 shows the interrelations of these devices. The active ingredient, 10, is loaded into the colloidal clay, 12, to yield a first control device or barrier, 14, which is a powder that can be used as is for controlling a target species. The strong binding of colloidal clay 12 provides a sustained release of active ingredient 10 from device 14. The release can be retarded by enrobing the powder particles with a recalcitrant polymer, 16, to form a control device barrier, 18, which is a polymer pellet. The enrobing process may include exfoliation of the active control agent sorbed nanoclay material. Barrier or polymer pellet 18 also may be easier and safer to handle than device 14. Alternatively, a third control device, 20, created when device 14 is loaded into a forming polymer, 22, and molded into a variety of useful shapes (e.g., fibers, films, slabs) that are described further below. Exfoliation of the active control agent sorbed nanoclay material can be done during this process. Device 24 is made by blending polymer pellets 18 with forming polymer 22 and molding them into products that have increased longevity and utility. Recalcitrant polymer 18 and forming polymer 22 can be formed from the same basic polymeric compositions, as listed below.

Each device (or barrier, these terms being used interchangeably to denote a physical structure that containing the loaded nanoclay for slow release of the loaded active control agent) can operate in one, two, or three dimensions. Examples of one-dimensional devices are filaments, strings, and cords. Examples of two-dimensional devices are coatings, films, sheets, and fabrics. Examples of three-dimensional devices are slabs, spray drops, and laminations. The device may be a continuous solid object or a discontinuous pattern of active ingredient loaded nanoclay or polymer pellets.

As stated above, the invention enables the artisan to place a barrier or other device at desired location for controlling unwanted pests for times ranging from days to months for certain target pests on up to years, e.g., 1 year, 10 years, 20 years, 30 years, or more, for other target pests. The barrier can be a fabric or other material loaded with the polymer pellets, a dispersal or pattern of the polymer pellets at the site; a coating, adhesive, caulk, sealant, or other material loaded with the polymer pellets, or the like. The precise form of the barrier is not the focus of the present invention, as a variety of barriers are known in the art. Lacking in the art is a method for providing sustained release of a control agent. By loading polymer pellets with colloidal clay loaded with the active ingredient or control agent, which sorbed colloidal clay-loaded polymer pellets are recalcitrant or retardant to release of the control agent and by judicious formulation of the barrier, effective sustained release of a control agent can be achieved for control of a target pest.

Insofar as the control agent or active ingredient is concerned, the nanoclay and polymer pellets do not distinguish between insecticides, insect repellents, attractants (e.g., sex pheromone and/or pheromone-like attractants), herbicides, fungicides, or the like, and mixtures thereof. Thus, the inventive system has broad applicability to a variety of pests. The same is true of the environment in which the pests can be found. That is, design of the polymer pellet and barrier design permits the sorbed colloidal clay-loaded polymer pellets to be used in desert environments, marine environments, home environments in virtually any climate, industrial and commercial environments, etc.

The colloidal state of the clay yields best performance in longevity and reduced control agent degradation, compared with conventional clay, carbon black, and other fillers proposed in the prior art. The Examples will detail such performance. Also, colloidal clays or nanoclays also are recognized as useful as a barrier to oxygen and other gases when blended with polymers. This characteristic adds to the longevity of the sorbed colloidal clay-loaded polymer pellets by retarding oxygen degradation of the control agent.

The length and breadth of colloidal particles have all three dimensions within the size range of about 0.5 nanometers to about 3000 nanometers. Exfoliated Nanocor I.30 E (see Examples), for example, is far from spherical. It has a thickness of about 1 nanometer and other dimensions of about 1500 nanometers. It is a miniature “flatland”. Broadly, then, colloidal particles for present purposes will range in size from about 20 microns to less than 1 nanometer. These dimensions result in extremely high average aspect ratios of around 200-500. For example, clays having an aspect ratio greater than about 50, thickness less than about 10 nanometers, and other dimensions greater than about 0.5 microns, find use in the present invention. An aspect ratio, then of the loaded nanoclays will range from between about 10:1 to about 1500:1 with a thin (narrowest) dimension of between about 0.1 nm and about 10 nm.

Sources of the nanoclays include, for example, a smectite, that is montmorillonite, or beidellite, or nonttronite, or saponite, or sauconite, or mixtures thereof or minerals with high percentage of smectite, such as bentonite. Alternatively, the colloidal clays can be derived from a vermiculite or illite. The colloidal clay may be loaded by melting a solid active ingredient and blending it with a smectite, for example, to make an expanded product, or by blending a fluid active ingredient with a smectite to make an expanded product.

Moreover, the small size and thickness means that one-gram contains over a million particles. Nanoclays take advantage of the effect of combining high aspect ratio and nanoscale size. Because nanometer-sized particles approach the scale of polymer molecules, a very close encounter can be made between the two materials when the clay is properly surface modified. The particle-molecule interaction creates a constrained region at the particle surface, which immobilizes a portion of the polymer matrix. With so many particles available for interactive association, the cumulative percent of constrained polymer can become large. In nylon polymer systems, for example, the constrained region exceeds 60% of the total matrix.

Beall, et. al. (U.S. Pat. No. 5,955,094) have shown how to intercalate layered clays with water or water/organic solvent mixtures. They have also shown how to displace with pesticides the water or water/organic solvent mixtures that are in the intercalated clays. Furthermore, they have shown how to exfoliate the pesticide intercalated clays. These exfoliated clays that contain pesticides are viscous liquids or gels that can be useful for relatively short duration release products. Their goal is to have all of the pesticide bound to individual platelets. Pesticide that did not end up on these platelets is a negative result.

The products of this invention have time horizons measured in years or decades. Therefore, solid products, not viscous liquids or gels, are needed. The solid preferably contains a mixture of pesticide species. Some of the pesticide is dissolved in the solid medium, some may be tiny pesticide crystals or droplets, some is trapped between layers of the clay (i.e., intercalated), and some is bound to tactoid species, and some to single platelets (exfoliated). This product is a dynamic system that evolves over the years. The evolving system is what generates the sustained release rates over decades of interaction with its environment.

With respect to the target organisms, the problem is that many organisms may be considered pets or benign under some circumstances, and pests under other circumstances. Frequently, a non-pest becomes a pest because it is in a location that humans define as inappropriate. The location may be acceptable, but the organism may be engaged in an activity that humans define as inappropriate. The damage done may be related to health, damage to property, esthetics, etc. Different cultures view certain organisms quite differently. Organisms fit into ecosystems, and exterminating a given species could have very undesirable overall results.

Thus, for present purposes, the invention defines “pest” in terms of:

1. species, and

2. location, and

3. activity to be controlled, and

4. damage to health/property/esthetics.

In the context of the present invention, the pest control agent emphasizes the location that prevents termites and other species from entering. A deer is a pest in this context, because a deer can facilitate a termite's breach of the barrier set up by the inventive system. A termite is not a pest if it is consuming dead wood in a forest. Roaches, termites, fire ants as well as clams, Zebra Mussels, and snails all play important and critical roles in the ecosystem; but in the wrong place relative to our homes and businesses (e.g., like generating energy in power plants or protecting our landscaping from the deer browsing on it), they are then condemned as a “pest”. Thus, organisms are undesirable in the human definition based upon damage to structures, materials, or reduction in yield of a desirable crop species by the invasion of an undesirable pest species into the farmers' field.

For present purposes, then, the terminology “pest species” will be used to identify those (unwanted) organisms that are to be controlled. That is, “pest termites” are termites that attack buildings. “Pest deer” invade our urban space. When termites and deer occupy their natural habitats, they are not “pests” for present purposes.

Pest species, then, can include, inter alia, microbes, fungi, algae, bacteria, viruses, spores, insects, birds, animals (land and sea), rodents, and the like. Specific such pest species include, inter alia, termites, ants, fire ants, mosquitoes, roaches, coffee bean borers, boring wasps, deer, squirrels, mice, rats, mollusks (clams, barnacles, mussels), and the like. For present purposes also, a “pesticide” is an active control agent or ingredient that repels, attracts, or kills pest species that are harmful. So long as the pest species does not invade the selected location, the control agent has accomplished its intended purpose, regardless of the mechanism of its action.

The sorption process can use pest control agent (e.g., diethyl adipate) vapor that contacts and permeates into the tiny clay particles. A fluidized bed process is especially convenient for loading the particles with volatile pest control agents. Molten pest control agent (e.g., Trifluralin) can be used for loading less volatile pest control agents.

The loaded colloidal clay is incorporated into a polymeric (e.g., elastomer) matrix that is advantageously is a polyurethane polymer. Other polymeric materials include, inter alia, polyethylene, polypropylene, polybutenes, natural rubber, polyisoprene, polyesters, styrene butadiene rubber, EPDM, polyacrylates, polymethacrylates, polyethylene terephthalate, polypropylene terephthalate, nylon 6, nylon 66, polylactic acid, polyhydroxy butyrate, polycarbonate, epoxy resins, or unsaturated polyester resins.

The pellet content of the system must contain enough active ingredient to release at a rate that is adequate to repel the target pest species for a period of time that meets the longevity goals. For example, if the release rate is one microgram/cm²/day for 30 years (ca. 11,000 days), then the pellets must store at least 11 mg for each square centimeter of surface area. The concentration of active ingredient in the bead additionally must not exceed a threshold level that would cause barrier failure.

Acceptable insecticides include those insecticides approved by the U.S. Environmental Protection Agency to kill or repel termites, ants, other boring insects, and wood decaying microorganisms. The class of insecticide which is presently preferred for use in the present invention are pyrethrins, including tefluthrin, bifenthrin, lambdacyhalothrin, cyfluthrin, deltamethrin, cypermethrin, permethrin, and natural permethrin. It will, however, be recognized by those skilled in the art that other effective insecticides such as isofenphos, fenvalerate, cypermethrin, organophosphate type insecticides, repellents as well as naturally occurring chemicals that act as irritants such as skunk oils and extracts of pepper can also be used. These insecticides are available from a number of commercial sources such as, for example, Dow Chemical Company, Bayer, ICI Industries, Velsicol, Novartus, Syngenta, and FMC Corporation.

Insecticides, pesticides, pest species repellents, alone or in combination with one and another, or in combination with other bioactive ingredients, such as fungicides, may also be used in accordance with the present invention. Combinations of insecticides, pesticides, repellents, nematicides (also referred to as nematocides), and fungicides additionally may be used to advantage. Fungicides include, for example, carboximide, dicarboximide, diflumetorim, ferimzone, chloropicrin, pentrachlorophenol, tri-chloronitromethane, 1-3 dichloropropane, and sodium N-methyl dithiocarbomate. Nematicides include 1,3 dichloropropene, ethoprophos, fenamiphos, benfuracarb, and cadusafos.

Commercial mollusicides include, inter alia: Niclosamide (Bayluscide) from Bayer; Clamtrol from Betz; Calgon H-130 from Calgon, and Mexel 432 from RTK Technologies. These products are intended for controlling Zebra Mussels that cause water intake problems for electric power plants and/or the snails that carry Schistosomiasis. Copper compounds, e.g., cuprous oxide, have been a favorite leachable component of antifouling paints. Insoluble cuprous chelates could be active ingredients that bloom to the surface and stay there repelling fouling organisms. Commercial antifouling paints (e.g., SIL MAR) that feature silicone ingredients make the surface too slippery for fouling fauna to form a stable attachment. Organotin compounds are known to work, but their use has been banned. Copper compounds are seen to present toxicity issues too. Organic antifouling agents, such as are disclosed in U.S. Pat. No. 5,441,743, may be used to advantage too. Endod, a natural plant extract from the soapberry bush, contains saponin and lemmatoxin. Endod has been used to control Zebra and Quagga Mussel infestations.

### Herbicide Embodiment

The root intrusion problems that affect sewer systems, sidewalks, golf courses, etc., can be treated effectively with herbicides. However, conventional sewer treatment only lasts for a few days or weeks. Most conventional systems for delivery of Trifluralin for appreciable lengths of time generate environmental burdens in the form of contamination of ground water. The few exceptions (e.g., BioBarrier I and II) are not suitable for application to pipes and other substrates of this invention; although, other systems can be adapted for use on irrigation and other piping.

Sustained release systems are needed, which systems keep the roots away for years. Trifluralin is an outstanding herbicide for these uses, but the suggested embodiments could be applied to other 2,6-dinitroaniline herbicides and many other types of herbicides. In this patent application, the term “TRIFLURALIN” includes other 2,6-dinitroaniline herbicides and other root-growth repellent herbicides.

While the invention has been described with reference to a preferred embodiment, those skilled in the art will understand that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. In this application all units are in the metric system and all amounts and percentages are by weight, unless otherwise expressly indicated. Also, all citations referred herein are expressly incorporated herein by reference.

### EXAMPLES

Various clay carriers were evaluated to determine their capacity to adsorb and retain pesticide, and their capacity to thereafter release the pesticide. The results recorded would apply to other control agents, e.g., herbicides, insect attractants, etc.

### Clays

(1) Attapulgus clay (ATTP)
(2) Montmorillinite (bentonite) clay
(3) Nanoclays from Nanocor, Inc. (onium ion amine modified montmorillonite products, intended for polymer use)
   (a) Nanomer I.30E (70%-75% Montmorillonite; 25%-30% protonated octadecylamine)
   (b) Nanomer I.30P (70%-75% Montmorillonite; 25%-30% protonated octadecylamine)
   (c) Nanomer I.34TCN (65%-80% Montmorillonite; 20%-35% methyl tallow bis(2-hydroxyethyl) ammonium salt
   (d) Nanomer I.44PA (77% Montmorillonite; 23%-30% dimethyl diialkyl
   [C14-C18] Ammonium salt
   (e) Nanomer PGV (100% Montmorillonite)

### Typical Mixing Procedure

Trifluralin (Treflan® from Dow Elanco; melting point reported to be 46°-47° C.) was heated to 68°-70° C., at which point it melted. A Blakeslee mixer (Model B-20) was adapted to have its interior heated to the desired temperature. The temperature of the clay, also 68°-70° C., and added pesticide within the bowl was maintained using heating straps attached to the outside mixing bowl (heaters controlled at 65° C., actual temp of stirred clay pesticide mixture was 50° C.). The nanoclay was slowly added to the mixer bowl at a rate of 5 mL/min-10 mL/min, with the mixer at a low (1) blending setting. Addition of the trifluralin was halted when the mixture just started to ball up. Mixing was continued for another hour at a higher mixing setting to break smaller clumps. The mixture then was cooled to room temperature, passed through a #60 sieve (<250 microns); remaining clumps (<10% total weight) were gently ground in a 1-quart Waring blender.

Liquid active ingredients (liquid at room temperature) were treated by the same procedure, except that the materials were not heated and cooled.

These procedures do not use water or organic solvents, as is customary in intercalating and exfoliating clays.

### Holding Capacity

Each tested active agent was slow-blended into the clay or nanoclay using a Blakeslee mixer, as described above. Active agents that were solid at room temperature were pre-melted, the clay heated, and the heated ingredients mixed by the same procedure. The following results were recorded:

TABLE 1

| | | HOLDING | |
|---|---|---|---|
| CLAY | ACTIVE AGENT | CAPACITY⁽¹⁾ | STATUS |
| ATTP | | | |
| 02-76-M | Dimethyl succinate | 0.5 | Liquid on surface |
| 02-76-N | Diethyl adipate | 0.48 | Good mix; swells |
| 02-76-P | 1-decanol | 0.43 | Good mix; swells |
| 02-76-Q | 2-methyl hexanoic acid | 0.42 | Good mix; swells |
| 02-76-R | 1,4-heptadienal | 0.39 | Good mix; swells |
| 02-76-S | Permethrin | 0.45 | No visible change |
| 02-76-T | Cypermethrin | 0.47 | No visible change |
| 02-76-U | Fenvalerate | 0.48 | No visible change |
| 02-76-X | Bifenthrin | 0.45 | No visible change |
| Bentonite | | | |
| 02-81-M | Dimethyl succinate | 0.52 | Liquid on surface |
| 02-81-N | Diethyl adipate | 0.48 | Good mix; swells |
| 02-81-P | 1-decanol | 0.46 | Good mix; swells |
| 02-81-Q | 2-methyl hexanoic acid | 0.39 | Good mix; swells |
| 02-81-R | 1,4-heptadienal | 0.38 | Good mix; swells |
| 02-81-S | Permethrin | 0.46 | No visible change |
| 02-81-T | Cypermethrin | 0.5 | No visible change |
| 02-81-U | Fenvalerate | 0.42 | No visible change |
| 02-81-X | Bifenthrin | 0.48 | No visible change |
| 02-81-Z | Trifluralin | 0.41 | Good mix; swells |
| Nanocor N | | | |
| I.34TCN | | | |
| 02-87-M | Dimethyl succinate | 0.67 | Good mix; swells |
| 02-87-P | 1-decanol | 0.62 | Good mix; swells |
| 02-87-S | Permethrin | 0.52 | Good mix; swells |
| 02-87-X | Bifenthrin | 0.51 | Good mix; swells |
| 02-87-Z | Trifluralin | 0.39 | Good mix; swells |
| Nanocor N I.44PA | | | |
| 02-87-Z | Trifluralin | 0.37 | Good mix; swells |
| Nanocor N I.30E | | | |
| 02-88-M | Dimethyl succinate | 0.69 | Good mix; swells |
| 02-8-N | Diethyl adipate | 0.27 | Good mix; swells |
| 02-88-P | 1-decanol | 0.27 | Good mix; swells |
| 02-88-S | Permethrin | 0.53 | Good mix; swells |
| 02-88-V | Nonanol | 0.55 | Good mix; swells |
| 02-88-X | Bifenthrin | 0.55 | Good mix; swells |
| 02-88-Z | Trifluralin | 0.46 | Good mix; swells |
| Nanocor N I.30P | | | |
| 02-89-M | Dimethyl succinate | 0.69 | Good mix; swells |
| 02-89-N | Diethyl adipate | 0.51 | Good mix; swells |
| 02-89-P | 1-decanol | 0.61 | Good mix; swells |
| 02-89-S | Permethrin | 0.56 | Good mix; swells |
| 02-89-V | Nonanol | 0.55 | Good mix; swells |
| 02-89-X | Bifenthrin | 0.53 | Good mix; swells |
| 02-89-Z | Trifluralin | 0.42 | Good mix; swells |
| Nanocor PGV | | | |
| 02-90-M | Dimethyl succinate | 0.45 | No visible change |
| 02-90-P | 1-decanol | 0.32 | Good mix; swells |
| 02-90-S | Permethrin | 0.4 | Liquid on surface |
| 02-90-V | Nonanol | 0.46 | Liquid on surface |
| 02-90-X | Bifenthrin | 0.41 | No visible change |
| 02-90-Z | Trifluralin | 0.44 | Liquid on surface |

⁽¹⁾Gm active/(gm active + clay) (e.g., 1/1 = 0.5, 2/1 = 0.66, 3/1 = 0.75, 4/1 = 0.8)

Thus, the sorption method of the present invention is applicable to a wide variety of solid and liquid active ingredients that have a variety of volatilities. Those that show no visible change may require more shearing to obtain exfoliation than the others.

The following table lists the average sorption for the various clays is as follows (the N I.44PA was ignored as only 1 active agent was tested) and the percentage increase of the nanoclays over the standard clays.

TABLE 2

| | | | % INCREASE |
|---|---|---|---|
| | AVERAGE | % INCREASE | OVER |
| CLAY TYPE | SORPTION | OVER ATTP | BENTONITE |
| ATTP | 0.452 | — | — |
| Bentonite | 0.410 | — | — |
| N I.34TCN | 0.542 | 19.9 | 32.20 |
| N I.30E | 0.474 | 4.87 | 15.61 |
| N I.30P | 0.553 | 22.3 | 34.88 |
| PGV | 0.413 | −8.63 | 0.73 |

Thus, the above-tabulated results indicate that the nanoclays have the capacity to sorb more active agent than conventional clays without use of water or organic solvents. Conventional sorption theory would predict that an increase in sorption capacity would be associated with higher release rates. That is, the additional active ingredient molecules would occupy clay surface sites that offer less firm binding. When the release rate data below are reviewed, the superiority of nanoclays to standard clays will be complete because the release rates are quite unexpectedly decreased!

A series of thermoplastic and thermoset materials were compounded with the pesticide adsorbed clay compositions and tested for their release rates. Lower release rates are preferred in order to extend the active life of the pesticidal composition. The following data were recorded.

### Thermoplastics

Injection molded samples (Table 3) were prepared using a Model 45 MINI-JECTOR (Mini-Jector Machinery Corp., Newbury, Ohio). The mold used produced test sheets that were 7.5×5 cm and 1 mm thick. The polyethylene used was powdered Quantum Microthene (XU594, 35 mesh). The polymer was mixed with the sorbent (clay or nanoclay) to provide a final ratio of 2 parts trifluralin to 20 parts polymer (24 gm load for each injection). For the PE, the injector was set up to melt the mixture at 127° C., with the injection nozzle heated to 138° C. Polypropylene was melted and injected at 163° C.

These sheets were washed in 90% MeOH to remove surface TFN contamination and placed into a flow device that exposes the sample to water that contains 0.01% Tween 20 and 0.5% MeOH. The system was operated at room temperature (ca. 23° C.). These conditions are used as an accelerated test in which 24 hours represents 2-3 years of exposure in the environment. During the first few hours, the release rate is high, usually over 100 μg/cm²/day. The test was continued until the release rate reaches the steady state that is reported in Table 3.

For extrusion and spinning of fibers, the trifluralin-loaded nanoclay was prepared from batches containing 3500 grams of trifluralin and 5476 grams of the nanoclay by the method described in Example 1. The particle size requirement was that the sample pass through a #60 U.S. Sieve (<250 microns). The loading of the trifuralin/nanoclay/polypropylene fiber material in a polyester matrix was adjusted to provide between 4 and 8% TFN (w/w) for the first fiber run, and 3% TFN (w/w) for the second test run. Pelletized PP material was used for the extrusion and spinning of fibers.

These samples were placed in a flow device that exposed the sample to water that contains 0.01% Tween 20. It was operated at room temperature (ca. 23° C.). These conditions are used as an accelerated test in which 24 hours represents 2 or 3 years of exposure in the environment. This correlation is based on the slope of Arrenhius plots over a temperature range of 10° C. to 50° C. I similar systems. During the first few hours, the release rate is high, usually over 100 μg/cm²/day. The test was continued until the release rate reaches the steady state that is reported in Table 3.

TABLE 3
Trifluralin Study

| | | RELEASE | |
|---|---|---|---|
| | | RATE | |
| | | (μG/ | |
| | | CM²/ | % NANOCLAY/ |
| SAMPLES | CLAY | DAY) | ATTP-PE |
| Polyethylene (MA 778-000) | ATTP | 17.49 | — |
| Polyethylene (MA 778-000) | PGV | 12.5 | 71 |
| Polyethylene (MA 778-000) | N I.44PA | 11.47 | 66 |
| Polyethylene (MA 778-000) | N I.30P | 7.73 | 44 |
| Polypropylene (MU 763-00) | ATTP* | — | — |
| Polypropylene (MU 763-00) | N PGV | 0.9 | 5 |
| Polypropylene (MU 763-00) | NI.44PA | 1.07 | 6 |
| Polypropylene (MU 763-00) | N I.30P | 0.41 | 2 |
| Polypropylene melt spun** | N I.30 P | 1-3 | 6-17 |

*Bad mixing/Extrusion
**Estimate in μg/cm²/day, because original data was recorded as 0.8-4 μg/g/day.

The release rates of nanoclay products were compared with conventional clay by calculation of the percentages shown in the right-hand column of Table 3. These results demonstrate that the nanoclays unexpectedly yield much lower release rates compared to standard clays. The extent of such lower release rates can be seen to range from 71% down to 44% for polyethylene and are even lower for polypropylene. Comparison of the rates for trifluralin release from polypropylene can be as low as 2% of that of the standard ATTP clay. It also shows that the choice of matrix polymer can make a significant difference in the relative release rates. The results for melt spinning experiments compared well with those done on molded sheets. These desirable results are due to the combination of the pesticide-loaded nanoclay and the matrix polymer. As stated above, the increased sorption capacity coupled with slower release rate makes the use of nanoclays for sustained release pesticide applications unexpected and unique.

### Thermoset Polymers

The following thermosets that contain N I.30E nanoclay loaded with a variety of pesticides were evaluated:

(a) Solithane S113, C113 and TIPApolyurethane (Uniroyal).

(b) Flexane 80 polyurea (ITW Devcon).

The pesticide-loaded clay or nanoclay was prepared by the method described in Example 1.

Solithane S113 is toluene diisocyanate (the isocyanate component) and C1134 is castor oil (the polyol component). The trifluralin-loaded N I.30E was dispersed into C113 and then blended with Solithane S113. Tripropanolamine (the catalyst) was added. These ingredients are mixed and cast into a mold that formed sheets similar to the ones used to evaluate the thermoplastics.

Flexane 80 liquid resin is an aliphatic diisocyanate (dicyclohexylmethane-4,4′-diisocyanate). Its curing agent is diethyl toluene diamine. The ratio of resin to curing agent was 78 to 22. The trifluralin-loaded N I.30E was blended with the curing agent and mixed with the resin. These ingredients are mixed and cast into a mold that formed sheets similar to the ones used to evaluate the thermoplastics.

The results of the release rate study are shown in Table 4. The release rate studies were performed as in Example 3. The poor results for decanol in the Solithane series were due to its reactivity with aromatic isocyanates.

The release rates for the urethanes are quite acceptable for most of the intended uses. They are not as low as the release rates from the experiments with thermoplastic polymers; however, both types could be optimized for higher or lower targets to meet target release rates.

TABLE 4*

| | | | | RELEASE |
|---|---|---|---|---|
| | | | | RATE |
| | | | | (μG/ |
| | | ACTIVE | | CM²/ |
| SAMPLE | POLYMER | AGENT | MIX/SET | DAY) |
| 02-79-M | Solithane/Urethane | Dimethyl | Good | 11 |
| | | succinate | | |
| | F80 Urethane | Dimethyl | Excellent | 22 |
| | | succinate | | |
| 02-79-N | Solithane/Urethane | Diethyl | Marginal | 14 |
| | | adipate | | |
| | F80 Urethane | Diethyl | Good | 16 |
| | | adipate | | |
| 02-79-P | Solithane/Urethane | 1-decanol | Poor | 31 |
| | F80 Urethane | 1-decanol | Good | 28 |
| 02-79-V | Solithane/Urethane | 1-Nonanol | Poor | 25 |
| | F80 Urethane | 1-Nonanol | Good | 19 |
| 02-79-S | Solithane/Urethane | Permethrin | Good | 5.7 |
| | F80 Urethane | Permethrin | Good | 5.9 |
| 03-11-X | Solithane/Urethane | Bifentrin | Good | 3.2 |
| | F80 Urethane | Bifentrin | Good | 4.5 |
| 03-11-T | Solithane/Urethane | Cypermethrin | Good | 6.5 |
| | F80 Urethane | Cypermethrin | Good | 7.9 |
| 03-11-U | Solithane/Urethane | Fenvalerate | Good | 6.1 |
| | F80 Urethane | Fenvalerate | Good | 6.4 |

Release rates measured with wipes of surfaces over 6-month period; active loading was set 10% parts by weight.
Release rates mimic vapor pressures, high release:high Vp

### Comparative Example

### Ruskin System for Release of Bioactives (TFN)

Ruskin (U.S. Pat. No. 5,116,414) describes the results of a study (FIG. 1) that is designed to demonstrate the efficacy on added I.30P nanoclay into a polymer containing a TFN and carbon black master batch. The commercial master batch material contains 25% TFN pre-sorbed to the carbon black (25%), and then is mixed with and incorporated into a polyethylene, and pellets produced by extrusion. Ruskin's reported results indicate that the added nanoclay results in a reduction of TFN release from 38 to 33 to 27 mg/sample/day for samples containing 0.6 and 5% free I.30P nanoclay, respectively. Reduction of release rates with added nanoclay was clearly shown, when the TFN is delivered from a sorbed TFN/carbon black reservoir.

### Testing

The present disclosure is quite distinct from that of Ruskin in that the molten active ingredient (e.g., TFN) is sorbed into a heated nanoclay (e.g., I.30P) and the material exfoliated in a molten polymer matrix (e.g., LLDPE). No carbon black is involved. No nanoclay that does not initially have sorbed active ingredient in it is involved. The Ruskin patent requires both of these ingredients.

A study was planned which included the Ruskin sample and an inventive set for comparison of release rate behavior. The master batch, called ROOTGUARD, was obtained from the manufacture/formulator, and the procedures followed in the Ruskin patent to obtain the blends that Ruskin had injection molded. Inventive samples also were injection molded, but not using a dripper mold. The results of the accelerated thermal test were not satisfactory (see FIGURE A). Several hypotheses could explain the difference between Ruskin's results and ours.
1) Ruskin's molding process prepares tortuous path, and the disclosed material does not.
2) Ruskin's LLDPE may be a different grade from ours, and he gives no hint which one to choose
3) Ruskin's exfoliation method may differ from ours.

It was decided to conduct some comparative experiments in which the same person uses the same methods and the same equipment. It also was decided to run accelerated experiments under less drastic conditions. Ruskin's samples were reproduced and an inventive set was included, using a set surface area, and a range of thermoplastic polymers.

The tests involved a comparative study at 41° and 31° C. until steady state release rates were attained. This also eliminated any projected problems with operating above the melting point of TFN.

Ruskin states that the formulation method of adding nanoclay to a system works for all LLDPE'S, and fails to indicate the specific LLDPE that was employed, we decided to compare a number of typical polymers that have been used to deliver bioactives/pesticides. Ethylene vinyl acetates and LDPE were not very attractive. It, therefore, was decided to use LLDPE and HDPE. The results are reported in Tables 1 and 2, below.

The Ruskin study employed the ROOTGUARD commercial master batch that is described in column 8 of Ruskin's patent. The BioGuard study employed a pre-loaded nanoclay that was prepared by absorbing pre-melted TFN (liquid) into heated I.30P nanoclay (all at 50°-55° C.). The final composition of the TFN/nanoclay composite was 42% TFN (w/w).

All measurements were gravimetric using a 5-place balance, with accuracy within 0.05 mg. Indicated samples were blended, held for no more than 2 hrs, then injection molded using a MINI-JECTOR Model 45. Six sequential injections were made for each formulation; only injection runs 3, 4, and 5 were used to select the 6 samples for gravimetric analysis. The injection mold produced 3 test rods per injection; these were rods with a length of 7.5 cm, a diameter of 0.63 cm, and a surface area of 14.9 cm². Three sample rods were employed in each of the 2 replicate cm, and a surface area of 44.8 cm². Three sample rods were employed in each of the 2 replicate cm, and a surface area of 29.7 cm². Three sample rods were employed in each of the 2 replicate sample groups per formulation. Samples were then placed in a forced air oven and held at 41±0.3° C. until steady state release was attained, and then transferred to 31±0.4° C. until steady state rates were attained. Samples were weighed at 2 or 3-day intervals.

In the first attempts to mimic the Ruskin procedures, the experiments employed test sheets with a high surface area to volume ratio, and depletion of TFN occurred in a few days. Thus we switched to the rod configuration, and lower operating temperatures.

### Study Results and Discussion

The formulations employed are indicated in Table 5. Samples 1 through 6 contained 6% TFN as employed in the Ruskin study. The Ruskin formulation uses the master batch with TFN pre-absorbed to XC-72 carbon black (25% TFN), while the BioGuard samples have the TFN pre-absorbed to the I.30P nanoclay (42% (w/w) TFN). Consistent with the procedure of Ruskin, neither the TFN or clay was pre-heated. The inventive formulation, however, was prepared in the manner described above where both the TFN and clay were heated before sorption.

Table 5: Mixing protocols for Ruskin/BGR&D comparative study to demonstrate the role of pre-loaded TFN/I.30P nanoclay prepared by BGR&D, and the influence of increasing free I.30P nanoclay as a secondary reservoir for TFN released from the master batch TFN contained in the carbon black carrier per Ruskin.

TABLE 5
Ruskin Formulations - 6% TFN per patent

| R | 1 | 2 | 3 |
|---|---|---|---|
| I.30P | 0 g | 7.5 g | 10.5 g |
| Indicated polymer | 37.5 g | 30 g | 27 g |
| Mix thoroughly | | | |
| RootGuard MB* | 12.5 | 12.5 | 12.5 g |

*Content of MB

| | |
|---|---|
| XC-72 CB | 25 g |
| LLDPE | 50 g |
| TFN | 25 g |

Inventive Formulations (g) - 6% TFN

| B4 | 4 | 5 | 6 |
|---|---|---|---|
| TFN/Clay (42% | 7.13 | 7.13 | 7.13 |
| load) | | | |
| I.30P | 8.25 g | 8.25 | 27.6 |
| TFN | 6 | 6 | 20 |
| Additional I.30P | 0 | 3.37 | 7.5 |
| Indicated | 42.82 | 39.4 | 35.33 |
| Polymer | | | |
| XC-72 CB | 0 | 0 | 0 |

Mix and hold samples for 2 hrs, injection mold

Table 2 provides the gravimetric data for the 6 formulations for the two polymers. Polymers are arranged from low melting points to high melting points. At 41° C., nanoclay additions result an increase in release rate for the 2 HDPE polymer. Only in the case of the formulation with LLDPE and LDPE do the release rates decline with added nanoclay; the only other formulation showing this trend is inventive formulation with LDPE. At 31° C., all formulations and polymers show either an increase in release rate, or no significant change with the added nanoclay. These data would indicate that there is little consistent influence of added nanoclay on the overall release rate from formulated polymers, whether the TFN is supplied from a pre-loaded carbon black or pre-loaded I.30P reservoir. It is also possible that the apparent increases noted in the Ruskin patent data with elevated temperatures (i.e., 56° C.). may have produced enough TFN from the carbon black reservoir to be forced into the unloaded nanoclay.

Even if the added nanoclay is being loaded as a secondary reservoir, there is no conceivable long-term delivery device using TFN or other bioactive that would have any functional longevity at this elevated temperature. Further it is presently unclear as to the kinetics of the TFN released from the carbon black reservoir, would the TFN remain in the polymer or penetrate the polymer-coated nanoclay.

Table 2: Release rate data for formulations and polymers. “Up, down or nt” noted for each formulation group indicates whether the addition of nanoclay increases, decreases or has no effect on release rate trend, respectively.

TABLE 6

| | | *Release Rate | *Release Rate |
|---|---|---|---|
| Polymer | Formulation | 41° C. | 31° C. |
| Exxon LL1002.09 | F1 | | |
| LLDPE | R2 | 301 ± 18 | 81 ± 1 |
| Mp 255 | R2 | 358 ± 75 down | 102 ± 16 up |
| Melt Flow 2 | R3 | 252 ± 28 | 114 ± 0 |
| Melt Flow 2 | B4 | 232 ± 8 | 53 ± 1 |
| Melt Flow 2 | B5 | 223 ± 11 nt | 64 ± 1 up |
| Melt Flow 2 | B6 | 226 ± 11 | 71 ± 1 |
| Dow 778-00 | | | |
| HDPE | R1 | 146 ± 32 | 16 ± 3 |
| Mp 325F | R2 | 247 ± 11 up | 24 ± 4 up |
| Melt Flow | R3 | 258 ± 7 | 28 ± 3 |
| | B4 | 102 ± 3 | 13 ± 1 |
| | B5 | 122 ± 6 up | 16 ± 1 nt |
| | B6 | 137 ± 2 | 14 ± 1 |

(μgTFN/cm2/day)

In performing this study it was noted on mixing of the components, particularly with the pelletized master batch, the polymer seemed to have a more rapid loading of TFN from the master batch pellets than the nanoclay. FIG. 1 shows a simple visualization, where a master batch pellet is placed in the center of a small plate and either LLDPE or I.30P packed around the pellet. After 4 hrs at 21° C., it can be observed that the TFN easily migrates to the un-melted polymer as well as the un-incorporated nanoclay.

It is very likely that other polymer parameters including percentage of amorphous regions, cross linking and especially exposed surface area, will affect the actual release rate from the delivery system employed. Thus our comparisons are likely reliable, based on the uncertainties in the Ruskin method. It appears that the addition of large quantities of nanoclay may in fact interfere with the polymer structure since in nearly all cases there is a noticeable increase in release rate with increasing clay content at elevated temperatures.

An important factor in considering these data is actual release rates. If the intention of the Ruskin study was to reduce release rate of his systems (and increase functional longevity) by adding un-loaded I.30P to a polymer containing a TFN-loaded carbon black, then the data do not support the supposition. Ruskin shows a 25% reduction in release rate by adding 21% un-loaded nanoclay to the carbon black system, compared with TFN/carbon black alone. While our data does not support this conclusion, it is clear from the data in Table 2 that the TFN/I.30P BioGuard system is superior in performance to the TFN/carbon back with 21% nanoclay or without added nanoclay. If we simply compare the 31° C. data (this temperature is at the upper extreme for most soil-buried delivery systems with TFN to attain any reasonable longevity), and look at just the LLDPE and the HDPE we see a pattern in performance. For the LLDPE, the BioGuard pre-loaded I.30P exhibits a 35% lower release rate than pre-loaded carbon black alone. The pre-loaded I.30P alone system exhibits a release rate that is 53% lower than for the Ruskin carbon black system containing an additional 21% I.30P. Similarly for HDPE, the BioGuard pre-loaded I.30P exhibits a 18% lower release rate than pre-loaded carbon black alone. The pre-loaded I.30P alone system exhibits a release rate that is 54% lower than for the Ruskin carbon black system containing an additional 21% I.30P.

We claim:

1. A method of forming an active control ingredient that controls target living species over an extended period of time, which comprises the steps of:
(a) heating a solid active control agent to a temperature of at least about 10° C. above the melting point of said solid active control agent; and
(b) loading an intercalated nanoclay retaining an ammonium ion chemical having 6 or more carbon atoms with said heated active control agent.

2. The method of claim 1, further comprising:
(c) loading a liquefied polymer with said loaded intercalated nanoclay, said polymer recalcitrant to release of said control ingredient.

3. The method of claim 1, further comprising:
(c) exfoliation said loaded intercalated nanoclay with a forming polymer.

4. The method of claim 1, wherein said intercalated nanoclay has a moisture content of less than 4%.

5. The method of claim 1, wherein said loaded nanoclay is exfoliated into particulates having an aspect ratio of between about 10:1 and about 1500:1, and a thin dimension of between about 0.1 nm and 10 nm.

6. The method of claim 1, wherein said active control ingredient is one or more of a chemical agent or a biological agent, which one or more of repels, attracts, kills, or exerts a desired action on said target living species.

7. The method of claim 1, wherein said nanoclay is derived by intercalation of one or more of a smectite, a vermiculite, or illite.

8. The method of claim 7, wherein said nanoclay is one or more of montmorillonite, beidellite, nonttronite, saponite, sauconite, or bentonite.

9. The method of claim 1, wherein the ammonium salt is one or more of protonated octadecyl amine, methyl tallow bis(2-hydroxyethyl) ammonium salt, or dimethyl diialkyl [C₁₄-C₁₈] ammonium salt.

10. The method of claim 1, further comprising:
(d) forming said loaded nanoclay into a barrier for control of said target living species.

11. A barrier for control of target living species, which comprises the active control ingredient made by the process of claim 1.

12. A barrier for control of target living species, which comprises the active control ingredient made by the process of claim 2.

13. A barrier for control of target living species, which comprises the active control ingredient made by the process of claim 3.

14. A barrier for control of target living species, which comprises the active control ingredient made by the process of claim 4.

15. A barrier for control of target living species, which comprises the active control ingredient made by the process of claim 5.

16. A barrier for control of target living species, which comprises the active control ingredient made by the process of claim 6.

17. A barrier for control of target living species, which comprises the active control ingredient made by the process of claim 7.

18. A barrier for control of target living species, which comprises the active control ingredient made by the process of claim 8.

19. A barrier for control of target living species, which comprises the active control ingredient made by the process of claim 9.
